(19) United States
(12) Patent Application Publication
(10) Pub. No.: US 2010/0216892 A1
(43) Pub. Date: Aug. 26, 2010

(54) Use of synergistically active 1,2-alkanediol mixtures as skin moisture-regulating compositions

(75) Inventors: Gerhard Schmaus, Hoxter-Bosseborn (DE); Ravikumar Pillai, Emerson, NJ (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Symrise GmbH & Co. KG, Holzminden (DE)

(21) Appl. No.: 11/721,113

(22) PCT Filed: Dec. 21, 2005
(86) PCT No.: PCT/EP05/57057

§ 371 Date: Jun. 7, 2007

Related U.S. Application Data
(60) Provisional application No. 60/639,910, filed on Dec. 29, 2004.

(51) Int. Cl. A61K 8/34; A61Q 19/00

(52) U.S. Cl. 514/738

(57) ABSTRACT

Synergistically active mixtures of straight-chain 1,2-alkanediols having 5 to 10 C atoms and their use as skin moisture-regulating compositions are described. Binary and ternary mixtures of 1,2-pentanediol, 1,2-hexanediol and 1,2-octanediol have proved to be particularly active here. Cosmetic or pharmaceutical formulations and ready-to-use cosmetic or pharmaceutical products comprising particularly synergistically active mixtures of the 1,2-alkanediols mentioned and the use of these formulations and products are furthermore described.

Synergistically active mixtures of straight-chain 1,2-alkanediols having 5 to 10 C atoms and their use as skin moisture-regulating compositions are described. Binary and ternary mixtures of 1,2-pentanediol, 1,2-hexanediol and 1,2-octanediol have proved to be particularly active here. Cosmetic or pharmaceutical formulations and ready-to-use cosmetic or pharmaceutical products comprising particularly synergistically active mixtures of the 1,2-alkanediols mentioned and the use of these formulations and products are furthermore described.

The skin is an important but at the same time also sensitive human organ, care of which is indispensable for physical and mental well-being. Numerous skin care compositions have been developed, which are available as creams, lotions, oils or gels and comprise specific skin care active compounds.

In addition to other aims of skin care, regulation of the water balance of the skin is of great importance, since skin tends to become damaged by drying out, last but not least due to influences of weathering and the environment. Mixtures in which the main constituents are often urea, free amino acids, pyrrolidonecarboxylic acid and lactate in various amounts are usually employed as moisture-regulating active compounds.

EP 0 655 904 discloses that 1,2-alkanediols having 5 to 10 C atoms have a very good skin-moisturizing action. It is furthermore disclosed that these substances at the same time have a very good skin tolerability and physiological acceptability and therefore can also be employed in a relatively high dosage as moisture-regulating active compounds in cosmetic products. EP 0 655 904 discloses in particular the use of alkanediols having 5 to 10 C atoms in cosmetic products, the use of straight-chain alkanediols having 5 to 7 C atoms, the OH groups of which are in the 1,2 position, being preferred. The amount of these alkanediols employed can be in the range of 1-10 wt. %, and is preferably 4-6 wt. %, in each case based on the cosmetic product. A particularly good skin moisture-regulating action has been credited to individual short-chain aliphatic 1,2-diols, and in fact in particular 1,2-pentanediol. The particularly good skin moisture-regulating property of 1,2-pentanediol is demonstrated with the aid of formulation examples. On the other hand, the use of diol mixtures comprising at least two aliphatic 1,2-diols for regulating skin moisture is not mentioned in EP 0 655 904.

WO 03/069994 discloses the use of 1,2-alkanediol mixtures comprising diols having 5-10 C atoms. However, particular attention is paid here to the use of 1,2-diol mixtures as antimicrobial active compound complexes in cosmetic and pharmaceutical products. The antimicrobial activity of the 1,2-diol mixtures described was clearly superior in this context to the activity of the individual diols at the same use concentration. The synergistic improvement in antimicrobial activity was demonstrated clearly in WO 03/069994 with the aid of Kull's equation. On the other hand, a synergistically intensified skin moisture-regulating action of 1,2-diol mixtures was not reported.

The search for suitable (active) substances which have an adequate skin-moisturizing action is made difficult for the person skilled in the art in that there is no clear dependency between the chemical structure of a substance or a substance mixture on the one hand and its skin moisture-regulating activity. Furthermore, there is no predictable connection between the skin moisture-regulating action, the toxicological acceptability, the skin tolerability and the stability of a substance or substance mixture.

On the basis of the statements in EP 0 655 904, it was surprising and unforeseeable that mixtures comprising or (substantially) consisting of at least two unbranched, i.e. straight-chain 1,2-alkanediols chosen from the group consisting of 1,2-pentanediol, 1,2-hexanediol, 1,2-heptanediol, 1,2-octanediol, 1,2-nonanediol and 1,2-decanediol have a significantly better, synergistically intensified skin moisture-regulating activity than the individual substances.

According to a first aspect, the invention therefore relates to the use of such a mixture comprising or consisting of two, three or more unbranched, i.e. straight-chain 1,2-alkanediols of different chain length chosen from the group consisting of 1,2-pentanediol, 1,2-hexanediol, 1,2-heptanediol, 1,2-octanediol, 1,2-nonanediol and 1,2-decanediol as a skin moisture-regulating composition.

In this context, it is preferable that the contents of the said diols in the mixture are adjusted such that their skin moisture-regulating action is synergistically intensified.

The use of a mixture comprising or consisting of

(a) 1,2-pentanediol and 1,2-hexanediol,
or
(b) 1,2-pentanediol and 1,2-octanediol,
or
(c) 1,2-hexanediol and 1,2-octanediol,
or
(d) 1,2-pentanediol, 1,2-hexanediol and 1,2-octanediol,
as a skin moisture-regulating composition is particularly preferred.

According to a second aspect, the invention relates to a composition comprising one of the mixtures (a), (b), (c) or (d) mentioned,

wherein
in alternatives (a), (b) and (c) the weight ratios of the two 1,2-alkanediols are preferably in the range of 3:2 to 2:3, preferably in the range of 45:55 to 55:45, and in alternative (c) preferably the weight ratio furthermore is not 1:1 since this ratio is known from WO 03/069994, and wherein
in alternative (d) the weight ratios of the three 1,2-alkanediols are preferably in the range of 25-40:25-40:25-40, preferably in the range of 30-35:30-35:30-35.

A composition according to the invention can be employed for regulating skin moisture.

A composition according to the invention (according to alternatives (a), (b), (c) and (d)), wherein the sum of the particular 1,2-alkanediols is at least 95 wt. %, preferably at least 98 wt. %, and particularly preferably at least 99 wt. %, based on the total weight of the composition, and the corresponding use according to the invention of these preferred compositions as skin moisture-regulating compositions is particularly preferred.

The contents of the particular 1,2-alkanediols in a mixture according to the invention or to be used according to the invention are preferably adjusted such that their skin moisture-regulating action is synergistically intensified.

The preferably synergistically active skin moisture-regulating 1,2-alkanediol mixtures according to the invention or to be used according to the invention have proved to be at the same time tolerated by the skin and physiologically acceptable; they can therefore also be employed in a comparatively high dosage as moisture-regulating active compounds in cosmetic or pharmaceutical products.

The preferably synergistically active, skin moisture-regulating 1,2-alkanediol mixtures according to the invention or to be used according to the invention which comprise exactly two 1,2-alkanediols chosen from the group consisting of 1,2-pentanediol, 1,2-hexanediol, 1,2-heptanediol, 1,2-octanediol, 1,2-nonanediol and 1,2-decanediol preferably comprise these two 1,2-alkanediols in a weight ratio in the range of from 10:1 to 1:10, preferably in the range of from 5:1 to 1:5, particularly preferably in the range of from 3:1 to 1:3, and very particularly preferably in the range of from 2:1 to 1:2. This applies in particular to mixtures according to alternatives (a), (b) and (c), see above.

The synergistically active, skin moisture-regulating 1,2-alkanediol mixtures according to the invention or to be used according to the invention which comprise three (or more than three) 1,2-alkanediols chosen from the group consisting of 1,2-pentanediol, 1,2-hexanediol, 1,2-heptanediol, 1,2-octanediol, 1,2-nonanediol and 1,2-decanediol preferably comprise the three 1,2-alkanediols (in the case of a mixture having more than three selected 1,2-alkanediols, the three 1,2-alkanediols with the highest substance content) in a weight ratio in the range of 1-10:1-10:1-10, preferably in the range of 1-5:1-5:1-5, particularly preferably in the range of 1-3:1-3:1-3, and very particularly preferably in the range of 1-2:1-2:1-2. This applies in particular to a mixture according to alternative (d), see above.

According to further aspects, the present invention relates to a cosmetic or pharmaceutical formulation or a cosmetic or pharmaceutical ready-to-use product (or the particular use thereof as a skin moisture-regulating composition) comprising a 1,2-alkanediol mixture according to the above alternatives (a), (b), (c) or (d), in particular in one of the preferred embodiments mentioned.

According to a further aspect, the present invention relates to the use of a 1,2-alkanediol mixture, for example a mixture according to the invention, which comprises two, three or more unbranched 1,2-alkanediols of different chain length chosen from the group consisting of 1,2-pentanediol, 1,2-hexanediol, 1,2-heptanediol, 1,2-octanediol, 1,2-nonanediol and 1,2-decanediol for the preparation of a skin moisture-regulating cosmetic or pharmaceutical product, the contents of the said 1,2-alkanediols preferably being adjusted such that their skin moisture-regulating action is intensified synergistically.

Preferred embodiments and further aspects of the present invention emerge from the following statements, the following examples and the attached patent claims.

The total amount of the abovementioned 1,2-alkanediol mixtures according to the invention or to be used according to the invention which is employed in ready-to-use cosmetic or pharmaceutical products is preferably in the range of 0.5-20 wt. %, preferably in the range of 1-10 wt. %, and particularly preferably in the range of from 2 to 8 wt %, in each case based on the ready-to-use cosmetic or pharmaceutical product.

The skin moisture-regulating 1,2-alkanediol mixtures according to the invention or to be used according to the invention can also be particularly advantageously combined here with further skin moisture-regulating substances. Cosmetic or pharmaceutical formulations and ready-to-use cosmetic or pharmaceutical products which comprise the synergistically active 1,2-alkanediol mixtures according to the invention or to be used according to the invention can therefore advantageously additionally comprise the following moisture-retaining regulators: sodium lactate, urea and derivatives, alcohols, glycerol, further diols, such as propylene glycol or hexylene glycol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulfate, lanolin, lanolin esters, amino acids, alpha-hydroxy acids (e.g. citric acid, lactic acid, malic acid) and derivatives thereof, mono-, di- and oligosaccharides, such as, for example, glucose, galactose, fructose, mannose, laevulose and lactose, polysugars, such as β-glucans, in particular 1,3-1,4-β-glucan from oats, alpha-hydroxy-fatty acids, triterpenic acids, such as betulinic acid or ursolic acid, and algae extracts.

The skin moisture-regulating 1,2-alkanediol mixtures according to the invention or to be used according to the invention can furthermore be employed together with osmolytes. Osmolytes which may be mentioned by way of example are: substances from the group consisting of sugar alcohols (myo-inositol, mannitol, sorbitol), quaternary amines, such as taurine, choline, betaine, betaine-glycine and ectoin, diglycerol phosphate, phosphorylcholine, glycerophosphorylcholines, amino acids, such as glutamine, glycine, alanine, glutamate, aspartate or proline, phosphatidylcholine, phosphatidylinositol and inorganic phosphates, as well as polymers of the compounds mentioned, such as proteins, peptides, poly-amino acids and polyols. All osmolytes at the same time have a skin-moisturizing action.

The skin moisture-regulating 1,2-alkanediol mixtures according to the invention or to be used according to the invention can be incorporated without difficulties into the usual cosmetic and/or dermatological and/or keratological formulations or ready-to-use products, such as, inter alia, pump sprays, aerosol sprays, creams, shampoos, ointments, tinctures, lotions, nail care products (e.g. nail varnishes, nail varnish removers, nail balsams) and the like. It is also possible here, and in some cases advantageous, to combine the synergistically active skin moisture-regulating 1,2-alkanediol mixtures with further active compounds. The cosmetic and/or dermatological and/or keratological formulations comprising synergistically active skin moisture-regulating 1,2-alkanediol mixtures can otherwise have the conventional composition here and serve for the treatment of skin and/or hair in the sense of a dermatological/keratological treatment or a treatment in the sense of care cosmetics. However, the synergistically active skin moisture-regulating 1,2-alkanediol mixtures can moreover also be employed in make-up products in decorative cosmetics.

Cosmetic formulations and ready-to-use cosmetic products which comprise skin moisture-regulating 1,2-alkanediol mixtures according to the invention or to be used according to the invention can moreover also comprise active compounds for preserving cosmetic products as well as antibacterial or antimycotic active compounds, perspiration-inhibiting active compounds (antiperspirants) and (metal) chelators.

For use, the cosmetic and/or dermatological and/or keratological formulations or ready-to-use products comprising skin moisture-regulating 1,2-alkanediol mixtures are applied to the skin and/or hair in a sufficient amount in the conventional manner for cosmetics and dermatics. In this context, cosmetic and dermatological formulations and ready-to-use cosmetic and dermatological products which comprise a 1,2-alkanediol mixture according to the invention or to be used according to the invention and additionally act as sunscreen agents also offer particular advantages. These formulations and products advantageously comprise at least one UVA filter and/or at least one UVB filter and/or at least one inorganic pigment. In this context, the formulations and ready-to-use cosmetic products can be in various forms such as are conventionally employed e.g. for this type of formulations and ready-to-use cosmetic products. They can be e.g. a solution, an emulsion of the water-in-oil (W/O) type or of the oil-in-water (O/W) type or a multiple emulsion, for example of the water-in-oil-in-water (W/O/W) type, a gel, a hydrodispersion, a solid stick or also an aerosol.

In cosmetic formulations and ready-to-use cosmetic products, the skin moisture-regulating 1,2-alkanediol mixtures according to the invention or to be used according to the invention can advantageously also be combined with cosmetic auxiliaries such as are conventionally used in such formulations and products, e.g. antioxidants, perfume oils, agents for preventing foaming, dyestuffs, pigments which have a colouring action, thickeners, surface-active substances, emulsifiers, softening substances, further moisturizing and/or moisture-retaining substances, fats, oils, waxes or other conventional constituents of a cosmetic formulation or ready-to-use product, such as alcohols, polyols, polymers, foam stabilizers, electrolytes, organic solvents or silicone derivatives. All conceivable antioxidants, perfume oils, agents for preventing foaming, dyestuffs, pigments which have a colouring action, thickeners, surface-active substances, emulsifiers, softening substances, moisturizing and/or moisture-retaining substances, fats, oils, waxes, alcohols, polyols, polymers, foam stabilizers, electrolytes, organic solvents or silicone derivatives which are suitable or usual for cosmetic and/or dermatological uses can be used here.

A high content of care substances is regularly advantageous in formulations or ready-to-use products which comprise skin moisture-regulating 1,2-alkanediol mixtures and are for topical prophylactic or cosmetic treatment of the skin. According to a preferred embodiment, the compositions comprise one or more fats and oils of animal and/or plant origin having care properties, such as olive oil, sunflower oil, refined soya oil, palm oil, sesame oil, rapeseed oil, almond oil, borage oil, evening primrose oil, coconut oil, shea butter, jojoba oil, sperm oil, beef tallow, neat's foot oil and lard, and optionally further care constituents, such as, for example, fatty alcohols having 8-30 C atoms. The fatty alcohols used here can be saturated or unsaturated and linear or branched.

Care substances which can be particularly preferably combined with the skin moisture-regulating 1,2-alkanediol mixtures according to the invention or to be used according to the invention moreover also include
ceramides, where ceramides are understood as meaning N-acylsphingosins (fatty acid amides of sphingosin) or synthetic analogues of such lipids (so-called pseudo-ceramides), which significantly improve the water retention capacity of the stratum corneum.
phospholipids, for example soya lecithin, egg lecithin and cephalins
vaseline, paraffin oils and silicone oils; the latter include, inter alia, dialkyl- and alkylarylsiloxanes, such as dimethylpolysiloxane and methylphenylpolysiloxane, as well as alkoxylated and quaternized derivatives thereof.

Animal and/or plant protein hydrolysates can advantageously also be added to the skin moisture-regulating 1,2-alkanediol mixtures according to the invention or to be used according to the invention. Substances which are advantageous in this respect are, in particular, elastin, collagen, keratin, milk protein, soya protein, oat protein, pea protein, almond protein and wheat protein fractions or corresponding protein hydrolysates, and also condensation products thereof with fatty acids and quaternized protein hydrolysates, the use of plant protein hydrolysates being preferred.

If a cosmetic or dermatological formulation comprising skin moisture-regulating 1,2-alkanediol mixtures according to the invention or to be used according to the invention is a solution or lotion, solvents which can be used are:
water or aqueous solutions;
fatty oils, fats, waxes and other natural and synthetic fat substances, preferably esters of fatty acids with alcohols of low C number, e.g. with isopropanol, propylene glycol or glycerol, or esters of fatty alcohols with alkanoic acids of low C number or with fatty acids;
alcohols, diols or polyols of low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

Mixtures of the abovementioned solvents are used in particular. In the case of alcoholic solvents, water can be a further constituent.

Cosmetic formulations and ready-to-use cosmetic products which comprise skin moisture-regulating 1,2-alkanediol mixtures according to the invention or to be used according to the invention can also be combined with one or more preservatives. Preservatives which are preferably chosen here are those such as benzoic acid, its esters and salts, propionic acid and its salts, salicylic acid and its salts, 2,4-hexadienoic acid (sorbic acid) and its salts, formaldehyde and paraformaldehyde, 2-hydroxybiphenyl ether and its salts, 2-zinc-sulfidopyridine N-oxide, inorganic sulfites and bisulfites, sodium iodate, chlorobutanolum, 4-ethylmercury-(II)5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury-(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxy-diphenyl ether, 4-chloro-3,5-dimethylphenol, 1,1′-methylene-bis(3-(1-hydroxymethyl-2,4-dioximidazolidin-5-yl)urea), poly-(hexamethylenediguariide) hydrochloride, 2-phenoxyethanol, hexamethylenetetramine, 1-(3-chloroallyl)-3,5,7-triaza-1-azonia-adamantane chloride, 1-(4-chlorophenoxy)-1-(1H-imidazol-1-yl)-3,3-dimethyl-2-butanone, 1,3-bis-(hydroxymethyl)-5,5-dimethyl-2,4-imidazolidinedione, benzyl alcohol, Octopirox, 1,2-dibromo-2,4-dicyanobutane, 2,2′-methylene-bis(6-bromo-4-chlorophenol), bromochlorophene, mixture of 5-chloro-2-methyl-3(2H)-isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxy-propan-2-ol, N-alkyl(C₁₂-C₂₂)trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxy-methylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)-dimethyl-benzyl-ammonium chloride, alkyl-(C₈-C₁₈)-dimethyl-benzylammonium bromide, alkyl-(C₈-C₁₈)-dimethyl-benzyl-ammonium saccharinate, benzyl hemiformal, 3-iodo-2-propynyl butylcarbamate, sodium hydroxymethyl-aminoacetate or sodium hydroxymethyl-aminoacetate.

Cosmetic formulations and ready-to-use cosmetic products which comprise skin moisture-regulating 1,2-alkanediol mixtures according to the invention or to be used according to the invention can also be particularly advantageously combined with cooling active compounds. Individual cooling active compounds or also a combination of several cooling active compounds, the use of which has proved to be particularly advantageous, are listed in the following, it being possible, however, for the list to be extended by any desired number of further cooling active compounds: l-menthol, d-menthol, racemic menthol, menthone glycerol acetal, menthyl lactate, substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxy-carboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and trioxaalkanoates, 3-menthyl methoxyacetate and icilin.

Cosmetic formulations and ready-to-use cosmetic products which comprise skin moisture-regulating 1,2-alkanediol mixtures according to the invention or to be used according to the invention can also particularly advantageously comprise antiinflammatory and/or redness- and/or itching-alleviating active compounds. All the antiinflammatory or redness- and/or itching-alleviating active compounds which are suitable or usual for cosmetic and/or dermatological uses can be used here. Antiinflammatory or redness- and/or itching-alleviating active compounds which are advantageously employed are steroidal antiinflammatory substances of the corticosteroid type, such as e.g. hydrocortisone, hydrocortisone derivatives, such as hydrocortisone 17-butyrate, dexamethasone, dexamethasone phosphate, methylprednisolone or cortisone, it being possible for the list to be extended by addition of further steroid antiinflammatories. Non-steroidal antiinflammatories can also be employed. There are to be mentioned here by way of example oxicams, such as piroxicam or tenoxicam; salicylates, such as aspirin, Disalcid, Solprin or fendosal; acetic acid derivatives, such as diclofenac, fenclofenac, indomethacin, sulindac, tolmetin or clindanac; fenamates, such as mefenamic, meclofenamic, flufenamic or niflumic; propionic acid derivatives, such as ibuprofen, naproxen or benoxaprofen, or pyrazoles, such as phenylbutazone, oxyphenylbutazone, febrazone or azapropazone. Alternatively, natural antiinflammatory substances or redness- and/or itching-alleviating substances can be employed. Plant extracts, specifically highly active plant extract fractions and highly pure active substances isolated from plant extracts, can be employed.

Extracts, fractions and active substances from camomile, aloe vera, Commiphora species, Rubia species, willow, rose-bay willow herb, oats, calendula, arnica, St. John's wort, honeysuckle, rosemary, Passiflora incarnata, witch hazel, Avena, Dianthus or Echinacea, as well as pure substances, such as, inter alia, bisabolol, apigenin, apigenin 7-glucoside, boswellic acid, phytosterols, glycyrrhizic acid, glabridin, licochalcone A and anthranilic acid amides, such as, in particular, avenanthramides or dianthramides, are particularly preferred. The formulations and ready-to-use products comprising synergistically active skin moisture-regulating 1,2-alkanediol mixtures can also comprise mixtures of two or more antiinflammatory active compounds.

Cosmetic formulations and ready-to-use cosmetic products which comprise skin moisture-regulating 1,2-alkanediol mixtures according to the invention or to be used according to the invention can also comprise antioxidants, it being possible for the antioxidants which are suitable or usual for cosmetic and/or dermatological uses to be used.

Cosmetic formulations and ready-to-use cosmetic products which comprise skin moisture-regulating 1,2-alkanediol mixtures according to the invention or to be used according to the invention can also comprise vitamins and vitamin precursors, it being possible for all the vitamins or vitamin precursors which are suitable or usual for cosmetic and/or dermatological uses to be used.

Cosmetic formulations and ready-to-use cosmetic products which comprise skin moisture-regulating 1,2-alkanediol mixtures according to the invention or to be used according to the invention can also comprise active compounds having a skin-lightening action. According to the invention, all the skin-lightening active compounds which are suitable or usual for cosmetic and/or dermatological uses can be used here. Advantageous skin-lightening active compounds in this respect are kojic acid, hydroquinone, arbutin, ascorbic acid, magnesium ascorbyl phosphate, liquorice root extracts and constituents thereof, glabridin or licochalcone A, or extracts of Rumex and Ramulus species, extracts from pine species (Pinus) or extracts from Vitis species which comprise, inter alia, skin-lightening stilbene derivatives.

Cosmetic formulations and ready-to-use cosmetic products which comprise skin moisture-regulating 1,2-alkanediol mixtures according to the invention or to be used according to the invention can also comprise active compounds having a skin-tanning action. According to the invention, all the skin-tanning active compounds which are suitable or usual for cosmetic and/or dermatological uses can be used in this respect. Dihydroxyacetone (DHA; 1,3-dihydroxy-2-propanone) may be mentioned here by way of example. DHA can be both in the monomeric and in the dimeric form, the content of dimers predominating in the crystalline form.

Cosmetic formulations and ready-to-use cosmetic products which comprise skin moisture-regulating 1,2-alkanediol mixtures according to the invention or to be used according to the invention can also comprise mono-, di- and oligosaccharides, such as, for example, glucose, galactose, fructose, mannose, laevulose and lactose.

Cosmetic formulations and ready-to-use cosmetic products which comprise skin moisture-regulating 1,2-alkanediol mixtures according to the invention or to be used according to the invention can also comprise plant extracts, which are conventionally prepared by extraction of the whole plant, but also in individual cases also exclusively from blossom and/or leaves, wood, bark or roots of the plant.

Cosmetic formulations and ready-to-use cosmetic products which comprise skin moisture-regulating 1,2-alkanediol mixtures according to the invention or to be used according to the invention can also comprise anionic, cationic, nonionic and/or amphoteric surfactants, in particular if crystalline or microcrystalline solids, for example inorganic micropigments, are to be incorporated into the formulations and ready-to-use cosmetic products. Surfactants are amphiphilic substances which can dissolve organic, nonpolar substances in water. In this context, the hydrophilic contents of a surfactant molecule are usually polar functional groups, for example —COO⁻, —OSO₃²⁻ or —SO₃⁻, while the hydrophobic parts as a rule are nonpolar hydrocarbon radicals. Surfactants are in general classified according to the nature and charge of the hydrophilic molecular moiety. A distinction can be made between four groups here:
anionic surfactants,
cationic surfactants,
amphoteric surfactants and
nonionic surfactants.

Anionic surfactants as a rule contain carboxylate, sulfate or sulfonate groups as functional groups. In aqueous solution, they form negatively charged organic ions in an acid or neutral medium. Cationic surfactants are almost exclusively characterized by the presence of a quaternary ammonium group. In aqueous solution, they form positively charged organic ions in an acid or neutral medium. Amphoteric surfactants contain both anionic and cationic groups and accordingly behave like anionic or cationic surfactants in aqueous solution, depending on the pH. In a strongly acid medium they have a positive charge, and in an alkaline medium a negative charge. On the other hand, they are zwitter-ionic in the neutral pH range. Polyether chains are typical of nonionic surfactants. Nonionic surfactants do not form ions in an aqueous medium.

### A. Anionic Surfactants

Anionic surfactants which are advantageously to be used are acylamino acids (and salts thereof), such as
acyl glutamates, for example sodium acyl glutamate, di-TEA-palmitoyl aspartate and sodium caprylic/capric glutamate,
acyl peptides, for example palmitoyl hydrolysed milk protein, sodium cocoyl hydrolysed soya protein and sodium/potassium cocoyl hydrolysed collagen,
sarcosinates, for example myristoyl sarcosine, TEA-lauroyl sarcosinate, sodium lauroyl sarcosinate and sodium cocoyl sarcosinate,
taurates, for example sodium lauroyl taurate and sodium methylcocoyl taurate,
acyl lactylates, for example lauroyl lactylate and caproyl lactylate
alaninates
   carboxylic acids and derivatives, such as
   for example, lauric acid, aluminium stearate, magnesium alkanolate and zinc undecylenate,
ester-carboxylic acids, for example calcium stearoyl lactylate, laureth-6 citrate and sodium PEG-4 lauramide carboxylate,
ether-carboxylic acids, for example sodium laureth-13 carboxylate and sodium PEG-6 cocamide carboxylate,
   phosphoric acid esters and salts, such as, for example, DEA-oleth-10 phosphate and dilaureth-4 phosphate,
   sulfonic acids and salts, such as
acyl isethionates, e.g. sodium/ammonium cocoyl isethionate,
alkylarylsulfonates,
alkylsulfonates, for example sodium coconut monoglyceride sulfate, sodium C₁₂₋₁₄ olefin-sulfonate, sodium lauryl sulfoacetate and magnesium PEG-3 cocamide sulfate,
sulfosuccinates, for example dioctyl sodium sulfosuccinate, disodium laureth-sulfosuccinate, disodium laurylsulfosuccinate and disodium undecylenamido-MEA-sulfosuccinate
   and
   sulfuric acid esters, such as
alkyl ether-sulfate, for example sodium, ammonium, magnesium, MIPA and TIPA laureth sulfate, sodium myreth sulfate and sodium C12-13 pareth sulfate,
alkyl sulfates, for example sodium, ammonium and TEA lauryl sulfate.
   B. Cationic surfactants

Cationic surfactants which are advantageously to be used are
alkylamines,
alkylimidazoles,
ethoxylated amines and
quaternary surfactants,
   RNH₂CH₂CH₂COO⁻ (at pH=7)
   RNHCH₂CH₂COO—B⁺ (at pH=12)=any desired cation, e.g. Na⁺
ester quats

Quaternary surfactants contain at least one N atom which is covalently bonded to 4 alkyl or aryl groups. This leads to a positive charge, independently of the pH. Alkylbetaine, alkylamidopropylbetaine and alkylamidopropylhydroxysulfaine are advantageous. The cationic surfactants used can furthermore preferably be chosen from the group consisting of quaternary ammonium compounds, in particular benzyltrialkyl-ammonium chlorides or bromides, such as, for example, benzyldimethylstearyl-ammonium chloride, furthermore alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether-sulfates, alkylpyridinium salts, for example lauryl- or cetylpyrimidinium chloride, imidazoline derivatives and compounds having a cationic character, such as amine oxides, for example alkyldimethylamine oxides or alkylaminoethyldimethylamine oxides. Cetyltrimethyl-ammonium salts in particular are advantageously to be used.

### C. Amphoteric Surfactants

Amphoteric surfactants which are advantageously to be used are
acyl-/dialkylethylenediamine, for example sodium acylamphoacetate, disodium acylamphodipropionate, disodium alkylamphodiacetate, sodium acylamphohydroxy-propylsulfonate, disodium acylamphodiacetate and sodium acylamphopropionate,
N-alkylamino acids, for example aminopropyl alkylglutarriide, alkylaminopropionic acid, sodium alkylimidodipropionate and lauroamphocarboxyglycinate.

### D. Nonionic Surfactants

Nonionic surfactants which are advantageously to be used are
alcohols,
alkanolamides, such as cocamides MEA/DEA/MIPA,
amine oxides, such as cocoamidopropylamine oxide,
esters which are formed by esterification of carboxylic acids with ethylene oxide, glycerol, sorbitan or other alcohols,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and coconut glycoside.
sucrose esters and ethers
polyglycerol esters, diglycerol esters, monoglycerol esters
methylglucose esters, esters of hydroxy acids

The use of a combination of anionic and/or amphoteric surfactants with one or more nonionic surfactants is furthermore advantageous.

The surface-active substance can be present in a concentration of between 1 and 98 wt. % in the formulations or ready-to-use cosmetic products comprising skin moisture-regulating 1,2-alkanediol mixtures, based on the total weight of the formulations or products.

Cosmetic or dermatological formulations and ready-to-use cosmetic or dermatological products which comprise skin moisture-regulating 1,2-alkanediol mixtures according to the invention or to be used according to the invention can also be in the form of emulsions.

The oily phase can advantageously be chosen from the following substance group:
mineral oils, mineral waxes
fatty oils, fats, waxes and other natural and synthetic fat substances, preferably esters of fatty acids with alcohols of low C number, e.g. with isopropanol, propylene glycol or glycerol, or esters of fatty alcohols with alkanoic acids of low C number or with fatty acids;
alkyl benzoates;
silicone oils, such as dimethylpolysiloxanes, diethylpolysiloxanes, diphenylpolysiloxanes and mixed forms thereof.

Compounds which can advantageously be employed are (a) esters of saturated and/or unsaturated branched and/or unbranched alkanecarboxylic acids having a chain length of from 3 to 30 C atoms and saturated and/or unsaturated, branched and/or unbranched alcohols having a chain length of from 3 to 30 C atoms, (b) esters of aromatic carboxylic acids and saturated and/or unsaturated, branched and/or unbranched alcohols having a chain length of from 3 to 30 C atoms. Preferred ester oils are isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, e.g. jojoba oil.

The oily phase can furthermore advantageously be chosen from the group consisting of branched and unbranched hydrocarbons and waxes, silicone oils and dialkyl ethers, the group consisting of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12 to 18 C atoms. The fatty acid triglycerides can advantageously be chosen from the group consisting of synthetic, semi-synthetic and natural oils, e.g. olive oil, sunflower oil, soya oil, groundnut oil, rapeseed oil, almond oil, palm oil, coconut oil, palm kernel oil and more of the like. Any desired blends of such oil and wax components can also advantageously be employed. In some cases it is also advantageous to employ waxes, for example cetyl palmitate, as the sole lipid component of the oily phase, and the oily phase is advantageously chosen from the group which consists of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicaprylyl ether. Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous. The hydrocarbons paraffin oil, squalane and squalene can also advantageously be used. The oily phase can furthermore advantageously have a content of cyclic or linear silicone oils or consist entirely of such oils, it nevertheless being preferable to use an additional content of other oily phase components in addition to the silicone oil or silicone oils. Cyclomethicone (e.g. decamethylcyclopentasiloxane) can advantageously be employed as a silicone oil. However, other silicone oils, for example undecamethylcyclotrisiloxane, polydimethylsiloxane and poly(methyl-phenylsiloxane), can also advantageously be used. Mixtures of cyclomethicone and isotridecyl isononanoate and of cyclomethicone and 2-ethylhexyl isostearate are furthermore particularly advantageous.

The aqueous phase of formulations or ready-to-use cosmetic products which comprise skin moisture-regulating 1,2-alkanediol mixtures according to the invention or to be used according to the invention and are in the form of an emulsion can advantageously comprise: alcohols, diols or polyols of low C number and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and furthermore alcohols of low C number, e.g. ethanol, isopropanol, 1,2-propanediol and glycerol, and, in particular, one or more thickeners, which can advantageously be chosen from the group consisting of silicon dioxide, aluminium silicates, polysaccharides and derivatives thereof, e.g. hyaluronic acid, xanthan gum and hydroxypropylmethylcellulose, particularly advantageously from the group consisting of polyacrylates, preferably a polyacrylate from the group consisting of the so-called Carbopols, for example Carbopols of the types 980, 981, 1382, 2984 and 5984, in each case individually or in combination.

Formulations and ready-to-use cosmetic products which comprise skin moisture-regulating 1,2-alkanediol mixtures according to the invention or to be used according to the invention and are in the form of an emulsion advantageously comprise one or more emulsifiers. O/W emulsifiers can advantageously be chosen, for example, from the group consisting of polyethoxylated or polypropoxylated or polyethoxylated and polypropoxylated products, e.g.:
the fatty alcohol ethoxylates
the ethoxylated wool wax alcohols,
the polyethylene glycol ethers of the general formula R—O—(—CH₂—CH₂—O—)ₙ—R′,
the fatty acid ethoxylates of the general formula R—COO—(—CH₂—CH₂—O—)ₙ—H,
the etherified fatty acid ethoxylates of the general formula R—COO—(—CH₂—CH₂—O—)ₙ—R′,
the esterified fatty acid ethoxylates of the general formula R—COO—(—CH₂—CH₂—O—)ₙ—C(O)—R′,
the polyethylene glycol glycerol fatty acid esters
the ethoxylated sorbitan esters
the cholesterol ethoxylates
the ethoxylated triglycerides
the alkyl ether carboxylic acids of the general formula R—COO—(—CH₂—CH₂—O—)ₙ—OOH,
wherein n represents a number from 5 to 30,
the polyoxyethylene sorbitol fatty acid esters,
the alkyl ether sulfates of the general formula R—O—(—CH₂—CH₂—O—)ₙ—SO₃—H
the fatty alcohol propoxylates of the general formula R—O—(—CH₂—CH(CH₃)—O—)ₙ—H
the polypropylene glycol ethers of the general formula R—O—(—CH₂—CH(CH₃)—O—)ₙ—R′
the propoxylated wool wax alcohols,
the etherified fatty acid propoxylates R—COO—(—CH₂—CH(CH₃)—O—)ₙ—R′
the esterified fatty acid propoxylates of the general formula R—COO—(—CH₂—CH(CH₃)—O—)ₙ—C(O)—R′
the fatty acid propoxylates of the general formula R—COO—(—CH₂—CH(CH₃)—O—)ₙ—H,
the polypropylene glycol glycerol fatty acid esters
the propoxylated sorbitan esters
the cholesterol propoxylates
the propoxylated triglycerides
the alkyl ether carboxylic acids of the general formula R—O—(—CH₂—CH(CH₃)—O—)ₙ—CH₂—COOH,
the alkyl ether sulfates and the acids on which these sulfates are based of the general formula R—O—(—CH₂—CH(CH₃)—O—)ₙ—SO₃—H,
the fatty alcohol ethoxylates/propoxylates of the general formula R—O—Xₙ—Yₘ—H
the polypropylene glycol ethers of the general formula R—O—Xₙ—Yₙ—Yₘ—R′
the etherified fatty acid propoxylates of the general formula R—COO —Xₙ—Yₘ—R′
the fatty acid ethoxylates/propoxylates of the general formula R—COO—Xₙ—Yₘ—H.

According to the invention, the polyethoxylated or polypropoxylated or polyethoxylated and polypropoxylated O/W emulsifiers employed are particularly advantageously chosen from the group consisting of substances having HLB values of 11-18, very particularly advantageously having HLB values of 14.5-15.5, if the O/W emulsifiers contain saturated radicals R and R′. If the O/W emulsifiers contain unsaturated radicals R and/or R′ or isoalkyl derivatives are present, the preferred HLB value of such emulsifiers can also be lower or higher.

It is of advantage to choose the fatty alcohol ethoxylates from the group consisting of ethoxylated stearyl alcohols, cetyl alcohols and cetyl stearyl alcohols (cetearyl alcohols). The following are particularly preferred:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isostea-reth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetyl stearyl ether (ceteareth-13), polyethylene glycol(14) cetyl stearyl ether (ceteareth-14), polyethylene glycol(15) cetyl stearyl ether (ceteareth-15), polyethylene glycol(16) cetyl stearyl ether (ceteareth-16), polyethylene glycol(17) cetyl stearyl ether (ceteareth-17), polyethylene glycol(18) cetyl stearyl ether (ceteareth-18), polyethylene glycol(19) cetyl stearyl ether (ceteareth-19) and polyethylene glycol(20) cetyl stearyl ether (ceteareth-20).

It is furthermore advantageous to chose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

Sodium laureth-11 carboxylate can advantageously be used as an ethoxylated alkyl ether carboxylic acid or salt thereof. Sodium laureth 1-4 sulfate can advantageously be used as an alkyl ether sulfate. Polyethylene glycol(30) cholesteryl ether can advantageously be used as an ethoxylated cholesterol derivative. Polyethylene glycol(25) sojasterol has also proved suitable.

The polyethylene glycol(60) evening primrose glycerides can advantageously be used as ethoxylated triglycerides.

It is furthermore advantageous to choose the polyethylene glycol glycerol fatty acid esters from the group consisting of polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caproate, polyethylene glycol(20) glyceryl oleate, polyethylene glycol(20) glyceryl isostearate, polyethylene glycol(18) glyceryl oleate/cocoate.

It is likewise favourable to choose the sorbitan esters from the group consisting of polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be employed are: fatty alcohols having 8 to 30 carbon atoms, monoglycerol esters of saturated and/or unsaturated, branched and/or unbranched alkanecarboxylic acids having a chain length of from 8 to 24, in particular 12 to 18 C atoms, diglycerol esters of saturated and/or unsaturated, branched and/or unbranched alkanecarboxylic acids having a chain length of from 8 to 24, in particular 12 to 18 C atoms, monoglycerol ethers of saturated and/or unsaturated, branched and/or unbranched alcohols having a chain length of from 8 to 24, in particular 12 to 18 C atoms, diglycerol ethers of saturated and/or unsaturated, branched and/or unbranched alcohols having a chain length of from 8 to 24, in particular 12 to 18 C atoms, propylene glycol esters of saturated and/or unsaturated, branched and/or unbranched alkanecarboxylic acids having a chain length of from 8 to 24, in particular 12 to 18 C atoms and sorbitan esters of saturated and/or unsaturated, branched and/or unbranched alkanecarboxylic acids having a chain length of from 8 to 24, in particular 12 to 18 C atoms.

W/O emulsifiers which are advantageous in particular are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyt alcohol, isobehenyl alcohol, selachyl alcohol, chimyl alcohol, polyethylene glycol(2) stearyl ether (steareth-2), glyceryl monolaurate, glyceryl monocaproate and glyceryl monocaprylate.

Reference may be made to the detailed statements in WO 03/069994 in respect of further cosmetic and pharmaceutical active compounds, base substances and auxiliary substances which can particularly preferably be combined with the skin moisture-regulating 1,2-alkanediol mixtures according to the invention or to be used according to the invention.

The skin moisture-regulating 1,2-alkanediol mixtures according to the invention or to be used according to the invention can also be employed as a constituent of fragrance compositions (odoriferous substance compositions, perfume) and, on the basis of their specific activity, for example, impart to a perfumed finished product an additional skin moisture-regulating property. A particularly preferred fragrance composition comprises (a) a sensorially active amount of a fragrance, (b) a skin moisture-regulating amount of a synergistically active 1,2-alkanediol mixture and (c) optionally one or more carrier substances and/or additives. Since the content of perfume in a cosmetic finished product is often in the region of approx. 1 wt. %, a perfume preferably comprises a synergistically active 1,2-alkanediol mixture according to the invention or to be used according to the invention to the extent of approx. 0.1-10 wt. %. The fact that the skin moisture-regulating 1,2-alkanediol mixtures according to the invention or to be used according to the invention have only a very weak intrinsic smell or are even completely odourless has proved to be particularly advantageous. As a result, they are suitable in particular for use in a fragrance composition, without thereby changing the fragrance properties of the fragrance composition in an adverse manner.

The invention is explained in more detail in the following with the aid of embodiment examples. Unless stated otherwise, the amounts data relate to the weight and the percentage data to the total weight of a particular mixture or product.

### EXAMPLE 1

### Human In Vivo Study to Demonstrate the Skin Moisture-Regulating properties of a 1,2-hexanediol/1,2-octanediol mixture (weight ratio: 1:1)

Because of its outstanding skin moisture-regulating properties, glycerol is very often employed as a skin moisture-regulating agent (“moisturizer”) in cosmetic formulations and ready-to-use cosmetic products. The skin moisture-regulating properties of a cosmetic formulation comprising a mixture according to the invention comprising 1,2-hexanediol and 1,2-octanediol (weight contents in the mixture in each case 50%) were therefore compared with those of a cosmetic formulation comprising glycerol.

### Test Procedure:

An oil-in-water emulsion (O/W cream) was employed as the base emulsion in the tests. 0.5 wt. % of the 1,2-alkanediol mixture comprising 1,2-hexanediol and 1,2-octanediol (ratio of amounts: 1:1; w/w) on the one hand (result: Test Emulsion 1) and 3 wt. % glycerol on the other hand (result: Test Emulsion 2) were added as skin moisture-regulating active compounds to batches of the base emulsion.

The influence of Test Emulsions 1 and 2 on the skin moisture was determined on test persons (volunteers) by means of corneometry; in addition, the transepidermal water loss was determined by means of a Tewameter.

Note: Corneometry is a capacitive measurement method in which the fact that the dielectric constant of water differs significantly from that of most other substances is utilized. A measurement capacitor (sensor) of appropriate shape reacts with different changes in capacitance, depending on the water content, on fields of skin to be investigated. These changes in capacitance of the sensor are processed fully automatically by the apparatus to give a digital measurement value. There is no conductive (galvanic) connection between the measurement object and the measuring apparatus; therefore almost no current flows through the measurement object. Properties such as ionic conductivity and polarization effects have practically no influence on the measurement result. The almost inertia-free “match” of the electronics to the moisture conditions found renders possible a very rapid measurement and substantial elimination of an influence on the results by involuntary movements or backing-up of moisture during the measurement.

For conditioning, the test persons were requested to use no skin moisture-regulating cosmetics for at least 1 week before the start of the studies. For cleansing purposes, the volunteers were allowed to use only a defined soap without skin moisture-regulating constituents over this period of time.

Immediately before the start of the actual tests, the test persons were acclimatized to a room temperature of 22° C. and 60% relative atmospheric humidity for 20 min.

The starting values of the skin moisture were then first measured both in a blank field and in the two test fields. The starting values in the blank field and test fields were determined per person.

Test Emulsions 1 and 2 were then each applied to one of the two test fields. Thereafter, exactly 15 and 30 minutes and 1, 2, 4 and 24 hours after application, the skin moisture was measured by means of corneometry and the transepidermal water loss (TEWL) by means of a Tewameter. The results are shown in the two tables which follow. The delta [%] values stated are the differences determined at the particular point in time between the particular product measurement value and the measurement value of the test field at time 0.

Corneometry—comparison of the delta [%] values of Test Emulsions 1 and 2 comprising 0.5 wt. % of a mixture of 1,2-hexanediol and 1,2-octanediol in the ratio of 1:1 w/w (Test Emulsion 1, called “S68” in the following table) or 3 wt. % glycerol (Test Emulsion 2, called “glycerol” in the following table):

| | Time | | | | | |
|---|---|---|---|---|---|---|
| | 15 min | 30 min | 1 h | 2 h | 4 h | 24 h |
| 0.5% S68 | 23.6 | 3 | 6.4 | 8.2 | 8.8 | 2.4 |
| 3% glycerol | 22.4 | 8.4 | 11.6 | 8.2 | 11 | 3.6 |

Tewameter measurements—comparison of the delta [%] values of test emulsions comprising 0.5 wt. % of a mixture of 1,2-hexanediol and 1,2-octanediol in the ratio of 1:1 w/w (Test Emulsion 1, called “S68” in the following table) or 3 wt. % glycerol (Test Emulsion 2, called “glycerol” in the following table):

| | Time | | | | | |
|---|---|---|---|---|---|---|
| | 15 min | 30 min | 1 h | 2 h | 4 h | 24 h |
| 0.5% S68 | −0.72 | −2.94 | −3.18 | −3.52 | −2.22 | −1.72 |
| 3% glycerol | −1.24 | −3.04 | −2.58 | −2.8 | −1.6 | −0.78 |

For the evaluation of the measurement results, it is decisive whether and, where appropriate, to what extent Test Emulsions 1 (“S68”) and 2 (“glycerol”) comprising 0.5% of diol mixture and, respectively, 3% of glycerol differ from one another in their values at the particular measurement time. As the measurements of the skin moisture state by means of corneometry and Tewameter measurement show, the two test emulsions give quite similar measurement values in respect of their skin moisture-regulating properties in the time window chosen. This means that the addition of only 0.5% of a 1,2-hexanediol/1,2-octanediol mixture to a base emulsion substantially has the same effect on skin moisture-regulating properties as the addition of 3% glycerol. An activity of the diol mixture (cf. Test Emulsion 1) which is better by a factor of approx. 6 compared with glycerol (cf. Test Emulsion 2) is thus demonstrated.

### EXAMPLE 2

### Human In Vivo Study to Demonstrate the Synergistically Intensified Skin Moisture-Regulating Activity of 1,2-Alkanediol Mixtures According to the Invention

An oil-in-water emulsion (O/W cream) was employed as the base emulsion in the tests. 1,2-Pentanediol, 1,2-hexanediol and 1,2-octanediol were chosen as skin moisture-regulating active compounds.

Starting from the base emulsion, 8 samples (test formulations) were prepared, cf. the following list. Samples A, B, C and D each comprised 6 wt. % of defined diol mixtures according to the invention, based on the end product. Samples E, F and G each comprised only one of the diols chosen from the group consisting of 1,2-pentanediol, 1,2-hexanediol and 1,2-octanediol in a concentration of 6 wt. %. Sample H, a placebo formulation, comprised no 1,2-diol.
A) 6 wt. % of a mixture according to the invention comprising in each case the same contents by weight of 1,2-pentanediol, 1,2-hexanediol and 1,2-octanediol (ratio: 1:1:1; w/w) in the base emulsion
B) 6 wt. % of a mixture according to the invention comprising in each case the same contents by weight consisting of 1,2-pentanediol and 1,2-octanediol (ratio: 1:1; w/w) in the base emulsion
C) 6 wt. % of a mixture according to the invention comprising 1,2-hexanediol and 1,2-octanediol (ratio: 1:1; w/w) in the base emulsion
D) 6 wt. % of a mixture according to the invention comprising 1,2-pentanediol and 1,2-hexanediol (ratio: 1:1; w/w) in the base emulsion
E) 6 wt. % 1,2-pentanediol in the base emulsion
F) 6 wt. % 1,2-hexanediol in the base emulsion
G) 6 wt. % 1,2-octanediol in the base emulsion
H) Pure base emulsion as a placebo formulation with a content of a 1,2-diol

The precise compositions of Test Formulations A to H can be seen from the attached Table A; in these, the base emulsion is phase B and the diols or diol mixtures are a constituent of phase A.

The influence on skin moisture by the 8 samples was determined in human in vivo experiments.

Test persons (volunteers) applied the sample to assigned test fields on the forearm twice daily for 14 days, a blank field remaining untreated for comparison.

In order to develop clearer results, winter test conditions were simulated with the aid of mild prior damage to the skin on the volar forearm. For this, the test persons washed the forearms with a 2% strength Na lauryl sulfate solution before each use of the sample. This procedure leads to drying out of the skin on the blank fields, and slight roughening also occurs. The extent to which these adverse effects are compensated by the test formulations is tested. Conclusive moisturizing values then result in comparison with the blank field.

The test persons were instructed to use no cosmetics on the forearms three days before the start of the test phase.

At the start of the test phase, the test persons were acclimatized to a room temperature of 22° C. and 60% relative atmospheric humidity for 45 min. The starting values of the skin moisture were then measured both in a blank field and in 8 test fields. The values of the test fields were determined per person. The skin moisture was measured by means of corneometry. Regarding this measurement method, see the notes above.

After using the samples for 14 days, a pause of approx. 12 h was incorporated, during which the test persons were also exposed to the Na lauryl sulfate treatment.

On the 15th day the final value of the skin moisture was measured, the procedure being the same as for the measurement of the starting value.

For evaluation of the measurement results, it is a matter of whether there is a significant difference in skin moisture between the starting value and the final value (in the corrected version) 12 hours after the last use of the samples. The measurement results show that a content of 6 wt. % of samples A, B, C and D comprising 1,2-alkanediol mixtures in the base emulsion has the effect of a significant improvement in the skin moisture both with respect to the action of the placebo sample H and with respect to the action of the particular samples E, F and G comprising only an individual diol. The improvement in the skin moisture due to samples A, B, C and D comprising in each case 6% (w/w) of diol mixtures according to the invention was significantly greater compared with samples E, F and G comprising in each case 6% (w/w) of individual 1,2-diols. This demonstrates that the 1,2-diol mixtures according to the invention or to be used according to the invention have the effect of a synergistically intensified improvement in skin moisture.

### EXAMPLE 3

### Ready-to-Use Cosmetic Products

Some synergistically active skin moisture-regulating 1,2-alkanediol mixtures comprising ready-to-use cosmetic products are listed by way of example in the following.

TABLE FOR EXAMPLE 3
FORMULATION EXAMPLES 1-7

| MATERIAL NAME | | % BY WEIGHT | | | | | |
|---|---|---|---|---|---|---|---|
| (SUPPLIER) | INCI | 1 | 2 | 3 | 4 | 5 | 7 |
| -(-Alpha-)-Bisabolol, | Bisabolol | | | 0.1 | | | |
| natural (Symrise) | | | | | | | |
| Abil 350 (Degussa- | Dimethicone | 0.5 | 2.0 | 1.0 | | | |
| Goldschmidt) | | | | | | | |
| Allantoin (Merck) | Allantoin | | | 0.1 | | | |
| Aloe Vera Gel Concentrate | Water (Aqua), Aloe | | | 3.0 | | | |
| 10/1 (Symrise) | Barbadensis Leaf Juice | | | | | | |
| Symatrix (Symrise) | Maltodextrin, Rubus | 0.3 | 0.1 | 1.0 | 0.1 | 0.3 | |
| | Fructicosus (Blackberry) Leaf | | | | | | |
| | Extract | | | | | | |
| Butylene glycol | Butylene Glycol | | | 5.0 | | | |
| Carbopol ETD 2050 | Carbomer | | | | | 0.2 | |
| (Noveon) | | | | | | | |
| Carbopol Ultrez-10 | Carbomer | | 0.1 | | | | |
| (Noveon) | | | | | | | |
| Cetiol OE (Cognis) | Dicaprylyl Ether | | | 4.0 | | | |
| Cetiol SB 45 (Cognis) | Butyrospermum Parkii (Shea | | | 1.0 | | | |
| | Butter) | | | | | | |
| Citric acid 10% strength | Citric Acid | | | | | | 0.3 |
| soln. | | | | | | | |
| Comperlan 100 (Cognis) | Cocamide MEA | | | | | | 0.5 |
| Dow Corning 246 Fluid | Cyclohexasiloxane (and) | | | | | 2.0 | |
| (Dow Corning) | Cyclopentasiloxane | | | | | | |
| Dow Corning 345 Fluid | Cyclomethicone | | | | 0.5 | | |
| (Dow Corning) | | | | | | | |
| D-Panthenol (BASF) | Panthenol | | | 1.0 | | | |
| Dracorin CE (Symrise) | Glyceryl Stearate Citrate | 5.0 | | | | | |
| Dracorin GMS (Symrise) | Glyceryl Stearate | | 2.0 | | | | |
| Dracorin GOC (Symrise) | Glyceryl Oleate Citrate, | | | | 2.0 | | |
| | Caprylic/Capric Triglyceride | | | | | | |
| Drago-Beta-Glucan | Water (Aqua), Butylene | 0.3 | | | | | |
| (Symrise) | Glycol, Glycerin, Avena | | | | | | |
| | Sativa (Oat) | | | | | | |
| | Kernel Extract | | | | | | |
| Dragocid Liquid (Symrise) | Phenoxyethanol, | | 0.80 | 0.70 | | 0.70 | |
| | Methylparaben, Ethylparaben, | | | | | | |
| | Butylparaben, Propylparaben, | | | | | | |
| | Isobutylparaben | | | | | | |
| Dragoderm (Symrise) | Glycerin, Triticum Vulgare | | | | | | 2.0 |
| | (Wheat) Gluten, Water | | | | | | |
| | (Aqua) | | | | | | |
| Drago-Oat-Active | Water (Aqua), Butylene | | | | 1.0 | | |
| (Symrise) | Glycol, Avena Sativa (Oat) | | | | | | |
| | Kernel Extract | | | | | | |
| Dragosan W/O P (Symrise) | Sorbitan Isostearate, | | | | | | |
| | Hydrogenated Castor Oil, | | | | | | |
| | Ceresin, Beeswax (Cera Alba) | | | | | | |
| Dragosantol (Symrise) | Bisabolol | | | | 0.1 | 0.1 | |
| Dragoxat EH (Symrise) | Ethylhexyl Ethylhexanoate | 3.0 | 3.0 | | 4.0 | | |
| EDETA B Powder (BASF) | Tetrasodium EDTA | | | | | | 0.1 |
| EDETA DB (BASF) | Disodium EDTA | | | | | 0.1 | |
| Emulsiphos (Symrise) | Potassium Cetyl Phosphate, | | 2.0 | | | 1.5 | |
| | Hydrogenated Palm | | | | | | |
| | Glycerides | | | | | | |
| Ethanol 96% | Ethanol | | | | | | |
| Extrapon Green Tea GW | Glycerin, Water (Aqua), | | 0.2 | | | | |
| (Symrise) | Camellia Sinensis Leaf | | | | | | |
| | Extract | | | | | | |
| Extrapone Hamamelis | Propylene Glycol, Hamamelis | | | | | | |
| Distillate, colourless | Virginiana (Witch Hazel) | | | | | | |
| (Symrise) | Water, Water (Aqua), | | | | | | |
| | Hamamelis Virginiana (Witch | | | | | | |
| | Hazel) Extract | | | | | | |
| Extrapon Camomile GW | Glycerin, Water (Aqua), | | 0.5 | | | | |
| (Symrise) | Chamomilla Recutita | | | | | | |
| | (Matricaria) Flower Extract | | | | | | |
| Extrapone Rosemary GW | Glycerin, Water (Aqua), | | 0.3 | | | | |
| (Symrise) | Rosmarinus officinalis | | | | | | |
| | (Rosemary) Leaf Extract | | | | | | |
| Frescolat ML cryst. | Menthyl Lactate | 0.5 | 0.5 | | 1.0 | 0.5 | 0.5 |
| (Symrise) | | | | | | | |
| Genapol LRO liquid | Sodium Laureth Sulfate | | | | | | 37.0 |
| (Clariant) | | | | | | | |
| Glycerin 85 P. | Glycerin | 3.0 | 2.0 | 4.0 | | 4.7 | |
| Urea | Urea | | | 0.5 | | | |
| Hydrolite-5 (1,2 | Pentylene Glycol | 3.0 | | 3.0 | 3.0 | | 3.0 |
| Pentanediol), Symrise | | | | | | | |
| 1,2 Hexanediol, Symrise | 1,2 Hexanediol | | 3.0 | 3.0 | | 3.0 | 3.0 |
| 1,2 Octanediol, Symrise | Caprylyl Glycol | 3.0 | 3.0 | | 3.0 | 3.0 | |
| Hydroviton (Symrise) | Water, Glycerin, Sodium | 0.5 | 1.0 | | 1.0 | 1.0 | 1.0 |
| | Lactate, TEA-Lactate, Serine, | | | | | | |
| | Lactic Acid, Urea, Sorbitol, | | | | | | |
| | Sodium Chloride, Lauryl | | | | | | |
| | Diethylenediaminoglycine, | | | | | | |
| | Lauryl Aminopropylglycine, | | | | | | |
| | Allantoin | | | | | | |
| Isodragol (Symrise) | Triisononanoin | | 2.0 | | | | |
| Isopropyl palmitate | Isopropyl Palmitate | 4.0 | | | | | |
| (Symrise) | | | | | | | |
| Keltrol RD (CP-Kelco) | Xanthan Gum | 0.2 | 0.1 | | | | |
| Keltrol T (Danby-Chemie) | Xanthan Gum | | | | | 0.2 | |
| Lanette 16 (Cognis) | Cetyl Alcohol | 1.0 | | | | | |
| Lanette O (Cognis) | Cetearyl Alcohol | | 3.0 | | | 1.0 | |
| Lara Care A-200 (Rahn) | Galactoarabinan | | | 0.3 | | | |
| Magnesium Sulfate | Magnesium Sulfate | | | | | | |
| (Merck) | | | | | | | |
| Menthol (Symrise) | Menthol | | | 0.2 | | | |
| Merquat 550 (Ondeo | Polyquaternium-7 | | | | | | 0.5 |
| Nalco) | | | | | | | |
| Sodium benzoate | Sodium Benzoate | | | | | | 0.5 |
| Neo Heliopan 357 | Butyl | | | | | 1.0 | |
| (Symrise) | Methoxydibenzoylmethane | | | | | | |
| Neo Heliopan AP | Disodium Phenyl | | | | | 4.6 | |
| (Symrise) | Dibenzimidazole | | | | | | |
| | Tetrasulfonate | | | | | | |
| Neo Heliopan AV | Ethylhexyl | | | | | 3.0 | |
| (Symrise) | Methoxycinnamate | | | | | | |
| Neo Heliopan Hydro | Phenylbenzimidazole Sulfonic | | | | | 6.7 | |
| (Symrise) | Acid | | | | | | |
| Neo Heliopan MBC | 4-Methylbenzylidene | | | | | 1.5 | |
| (Symrise) | Camphor | | | | | | |
| Neo Heliopan OS | Ethylhexyl Salicylate | | | | | 5.0 | |
| (Symrise) | | | | | | | |
| Neutral oil | Caprylic/Capric Triglyceride | 6.0 | | | 4.0 | 2.0 | |
| Paraffin oil 5 degree E | Paraffinum Liquidum | | | | 4.0 | | |
| (Parafluid) | | | | | | | |
| PCL Liquid 100 (Symrise) | Cetearyl Ethylhexoate | 3.0 | 5.0 | | 7.0 | | |
| PCL Solid (Symrise) | Stearyl Heptanoate, Stearyl | | 2.0 | | | | |
| | Caprylate | | | | | | |
| PCL-Liquid (Symrise) | Cetearyl Ethylhexanoate, | | 1.0 | | | | |
| | Isopropyl Myristate | | | | | | |
| Pemulen TR-2 (Noveon) | Acrylates/C10-30 Alkyl | | | 0.3 | 0.2 | | |
| | Acrylate Crosspolymer | | | | | | |
| Propylene glycol-1,2 99P | Propylene Glycol | | 5.0 | | | | |
| GC | | | | | | | |
| Sodium chloride | Sodium Chloride | | | | | | 1.0 |
| Sodium hydroxide (10% | Sodium Hydroxide | | 0.3 | 0.6 | 0.4 | | |
| strength soln. | | | | | | | |
| Sweet almond oil (Wagner) | Prunus dulcis | 0.5 | | | | | |
| Perfume oil (Symrise) | Fragrance | 0.3 | 0.3 | 0.3 | 0.2 | 0.4 | 0.5 |
| Tego Betaine L7 (Degussa) | Cocamidopropyl Betaine | | | | | | 6.0 |
| Tegosoft TN (Degussa) | C12-15 Alkyl Benzoate | | | 5.0 | | 5.0 | |
| Triethanolamine, 99% | Triethanolamine | | | | | 0.5 | |
| Retinyl Palmitate in Oil | Retinyl Palmitate | 0.05 | | | | | |
| (DSM Nutritional Products) | | | | | | | |
| Tocopherol Acetate (DSM | Tocopheryl Acetate | | | 0.5 | | 0.5 | |
| Nutritional Products) | | | | | | | |
| Water, demineralized | Water (Aqua) | to 100 | to 100 | to 100 | to 100 | To 100 | to 100 |

1 = Moisturizing cream O/W
2 = Moisturizing skin lotion O/W, with plant extract
3 = Moisturizing after-sun balm
4 = Body spray, for moisture-deficient skin
5 = Sunscreen lotion (O/W), broadband protection
7 = Shampoo

TABLE A for Example 2
Composition of Test Formulations A to H

| | | | | | | | | | H |
|---|---|---|---|---|---|---|---|---|---|
| Phase A wt. % | INCI | A | B | C | D | E | F | G | (placebo) |
| Water | Water (Aqua) | to 100 | to 100 | to 100 | to 100 | to 100 | to 100 | to 100 | to 100 |
| Carbopol ETD 2050 | Carbomer | 0.15 | 0.15 | 0.15 | 0.15 | 0.15 | 0.15 | 0.15 | 0.15 |
| Hydrolite-5 (1,2 | Pentylene Glycol | 2.0 | 3.0 | — | 3.0 | 6.0 | — | — | — |
| pentanediol) | | | | | | | | | |
| 1,2-Hexanediol | 1,2-Hexanediol | 2.0 | — | 3.0 | 3.0 | — | 6.0 | — | — |
| 1,2-Octanediol | Caprylyl Glycol | 2.0 | 3.0 | 3.0 | — | — | — | 6.0 | — |
| Phase B | | | | | | | | | |
| Dracorin CE | Glyceryl Stearate/Citrate | 3.0 | 3.0 | 3.0 | 3.0 | 3.0 | 3.0 | 3.0 | 3.0 |
| Emulsiphos | Potassium Cetyl | 1.0 | 1.0 | 1.0 | 1.0 | 1.0 | 1.0 | 1.0 | 1.0 |
| | Phosphate, | | | | | | | | |
| | Hydrogenated Palm | | | | | | | | |
| | Glycerides | | | | | | | | |
| Dragoxat EH | Ethylhexyl Ethylhexanoate | 7.5 | 7.5 | 7.5 | 7.5 | 7.5 | 7.5 | 7.5 | 7.5 |
| Isodragol | Triisononanoin | 4.0 | 4.0 | 4.0 | 4.0 | 4.0 | 4.0 | 4.0 | 4.0 |
| Dracorin GMS | Glyceryl Stearate | 2.0 | 2.0 | 2.0 | 2.0 | 2.0 | 2.0 | 2.0 | 2.0 |
| Lanette O | Cetearyl Alcohol | 3.0 | 3.0 | 3.0 | 3.0 | 3.0 | 3.0 | 3.0 | 3.0 |
| Abil 350 | Dimethicone | 0.5 | 0.5 | 0.5 | 0.5 | 0.5 | 0.5 | 0.5 | 0.5 |
| Phase C | | | | | | | | | |
| Sodium hydroxide 10% | Sodium Hydroxide | 0.3 | 0.3 | 0.3 | 0.3 | 0.3 | 0.3 | 0.3 | 0.3 |

pH: 5.8-6.0

### Preparation Instructions:

Disperse Carbopol ETD 2050 in water using an Ultra Turrax stirrer (UTR).

Heat phases A and B separately to approx. 80° C.

Add phase B to phase A and homogenize. (UTR, 2 min, 5,000 rpm)

Subsequently add phase C and stir the emulsion until cold, down to 35° C., using a blade stirrer.

Note: The various diols are added to the aqueous phase.

Depending on the concentration, the water content is reduced accordingly.

1. A method of preparing a skin moisture-regulating composition comprising mixing two, three or more unbranched 1,2-alkanediols of different chain length chosen from the group consisting of 1,2-pentanediol, 1,2-hexanediol, 1,2-heptanediol, 1,2-octanediol, 1,2-nonanediol and 1,2-decanediol to a product.

2. A method according to claim 1, wherein said diols are adjusted such that their skin moisture-regulating action is synergistically intensified.

3. A method according to claim 1 wherein said composition comprises
(a) 1,2-pentanediol and 1,2-hexanediol,
or
(b) 1,2-pentanediol and 1,2-octanediol,
or
(c) 1,2-hexanediol and 1,2-octanediol,
or
(d) 1,2-pentanediol, 1,2-hexanediol and 1,2-octanediol,
as a skin moisture-regulating composition.

4. A method according to claim 1 wherein said composition comprises two 1,2-alkanediols, wherein the weight ratio of the two 1,2-alkanediols is in the range of from 10:1 to 1:10.

5. A method according to claim 1 wherein said composition comprises three 1,2-alkanediols, wherein the weight ratio of the three 1,2-alkanediols is in the range of 1-10:1-10:1-10.

6. A method according to claim 1 wherein said composition comprises a 1,2-alkanediol mixture which comprises two, three or more unbranched 1,2-alkanediols of different chain length chosen from the group consisting of 1,2-pentanediol, 1,2-hexanediol, 1,2-heptanediol, 1,2-octanediol, 1,2-nonanediol and 1,2-decanediol for the preparation of a skin moisture-regulating cosmetic or pharmaceutical product.

7. A composition comprising
(a) 1,2-pentanediol and 1,2-hexanediol, or
(b) 1,2-pentanediol and 1,2-octanediol, or
(c) 1,2-hexanediol and 1,2-octanediol, or
(d) 1,2-pentanediol, 1,2-hexanediol and 1,2-octanediol,
wherein in alternatives (a) and (b) the weight ratios of the two 1,2-alkanediols are in the range of from 3:2 to 2:3, in alternative (c) the weight ratios of the two 1,2-alkanediols are in the range of 3:2 to 2:3, and
wherein in alternative (d) the weight ratios of the three 1,2-alkanediols is in the range of 25-40:25-40:25-40.

8. A composition according to claim 7, wherein the sum of the 1,2-alkanediols is at least 95 wt. %, based on the total weight of the composition.

9. A cosmetic or pharmaceutical formulation or cosmetic or pharmaceutical ready-to-use product comprising a composition according to claim 7.

10. A method for formulating a cosmetic or pharmaceutical formulation or cosmetic or pharmaceutical ready-to-use product as a skin moisture-regulating composition comprising adding a composition according to claim 7 to said cosmetic or pharmaceutical formulation or said cosmetic or pharmaceutical ready-to-use product.
